(19) United States
(12) Patent Application Publication
(10) Pub. No.: US 2025/0361222 A1
(43) Pub. Date: Nov. 27, 2025

(54) Extraction of nicotine from tobacco by closed cycle steam stripping and scrubbing

(71) Applicant: Leopold Samulis, York (GB)

(72) Inventor: Leopold Samulis, York (GB)

(21) Appl. No.: 19/288,991

(22) Filed: Aug. 2, 2025

(51) Int. Cl. C07D 401/04; A24B 15/24; B01D 53/14

(52) U.S. Cl. CPC C07D 401/04; A24B 15/243; B01D 53/1487; B01D 2257/70

(57) ABSTRACT

A method of extracting nicotine or other alkaloids from an alkaloid-containing feedstock by continuously passing steam around a closed circuit wherein it is alternately contacted with the alkaloid-containing feedstock, and a material capable of capturing the target alkaloid from the steam. By avoiding the need for continuous evaporation and condensation, the energy requirements are vastly reduced compared to classic steam distillation or steam distillation using multi-effect evaporators or mechanical vapor recompression (MVR). Operating at atmospheric pressure improves safety and simplifies equipment construction, while environmental sustainability is achieved by not requiring any solvents, gases, or other chemicals beyond simple acids and bases such as sulfuric acid and calcium hydroxide.

PRIOR ART

| | | |
|---|---|---|
| U.S. Pat. No. 678,362A | 1901 Jul. 16 | Pascal Davie |
| U.S. Pat. No. 2,128,043A | 1938 Aug. 23 | Hope Natural Gas Co |
| U.S. Pat. No. 2,162,738A | 1939 Jun. 20 | Clarence E Mccoy |
| U.S. Pat. No. 2,227,863A | 1941 Jan. 7 | Kinetic Chemicals Inc |
| U.S. Pat. No. 3,046,997A | 1962 Jul. 31 | Philip Morris USA Inc |
| U.S. Pat. No. 3,803,004A | 1974 Apr. 9 | Tamag Basel AG |
| U.S. Pat. No. 4,153,063A | 1979 May 8 | Studiengesellschaft Kohle gGmbH |
| U.S. Pat. No. 5,018,540A | 1991 May 28 | Philip Morris USA Inc |
| U.S. Pat. No. 5,119,835A | 1992 Jun. 9 | British American Tobacco Germany |
| | | GmbH |
| U.S. Pat. No. 5,148,819A | 1992 Sep. 22 | R J Reynolds Tobacco Co |
| U.S. Pat. No. 5,197,494A | 1993 Mar. 30 | R J Reynolds Tobacco Co |
| U.S. Pat. No. 8,887,737B2 | 2014 Nov. 18 | Philip Morris USA Inc |
| U.S. Pat. No. 10,610,526B2 | 2020 Apr. 7 | Next Generation Labs LLC |
| U.S. Pat. No. 12,075,810B2 | 2024 Sep. 3 | Nicoventures Trading Ltd |
| CN113582972A | 2023 Mar. 28 | Shenzhen Zinwi Biotech Co Ltd |

ISO 2881: 1992 Tobacco and tobacco products - Determination of alkaloid content - Spectrophotometric method

### BACKGROUND OF THE INVENTION

Nicotine is a mild stimulant found naturally in the tobacco plant. After isolation as a pure chemical, it is used in the manufacture of pesticides, medicines, smoking cessation aids, and liquids for electronic cigarettes.

Although nicotine can be produced synthetically from purely chemical feedstock (U.S. Pat. No. 10,610,526, CN 113,582,972), the majority of nicotine on the market today is extracted from the tobacco plant. Plant-sourced nicotine is preferred for its lower cost of production, naturally high enantiomeric purity, presence of trace alkaloid impurities that add flavor to e-cigarettes, and the ability to certify the nicotine as being naturally-sourced.

Historically, nicotine has been extracted from the tobacco plant either by steam distillation or by a solvent extraction process, in which the solvent may be liquid phase (U.S. Pat. No. 678,362, U.S. Pat. Nos. 2,128,043, 2,162,738, 2,227,863, 3,046,997), gas phase (U.S. Pat. No. 5,197,494), or supercritical (U.S. Pat. Nos. 4,153,063, 5,018,540, 5,119,835, 8,887,737, 12,075,810), or by an aqueous extraction process in which an organic solvent is usually used to recover the nicotine from the aqueous phase (U.S. Pat. No. 5,148,819).

Although steam distillation is well understood and suitable for laboratory assay (U.S. Pat. No. 3,803,004, ISO 2881:1992), it is not employed commercially for nicotine production due to the extreme energy costs required for evaporating water and re-condensing steam.

Solvent extraction, wherein alkalized plant material is contacted with organic solvents such as petroleum ether, remains the dominant commercial process. While economical, it suffers from many disadvantages, including: factory safety issues due to solvent flammability and toxicity, environmental problems from solvent vapors and residual solvent in the waste tobacco, consumer safety issues due to traces of solvent remaining in the final product, and the inability to certify the nicotine as organic and chemical-free.

While using supercritical extraction with non-toxic solvents such as carbon dioxide solves some of these issues, the inherent high pressures increase the complexity and cost of fabricating and maintaining plant equipment and create additional safety hazards.

### SUMMARY OF THE INVENTION

This patent introduces a method of extracting nicotine from tobacco that is reminiscent of steam distillation, yet vastly reduces energy requirements by eliminating the need for continuous evaporation and condensation.

The invention consists of a closed system, wherein a steam phase travels continuously between a stripper and a scrubber. In the stripper, the steam phase is contacted with a liquid phase consisting of alkalized tobacco dust in water, whereupon a portion of the nicotine evaporates from the liquid phase into the steam phase. In the scrubber, the nicotine-containing steam phase is contacted with a solid or liquid absorbent that captures and retains the nicotine. To prevent re-release of nicotine from the absorbent and to drive the whole system equilibrium towards the desired outcome, it is preferable to use a medium that irreversibly captures nicotine, such as a strongly acidic medium that irreversibly binds the nicotine as a salt. To complete the cycle, the steam phase is then returned to the stripping section whereupon it may once again absorb more nicotine. As the cycle continues, the nicotine concentration in the alkalized tobacco sludge approaches zero, while the concentration in the absorbent phase increases. The process is deemed finished when the economic costs of extracting further nicotine outweighs the benefits. An extraction efficiency of 95˜98% vs. total laboratory nicotine assay is typically obtained.

When compared to classic steam distillation, the novelty lies in the retention of all the benefits of steam distillation, such as environmentally friendly materials, moderate temperatures and pressures, and benign waste products, while vastly reducing the energy requirements. When compared to steam distillation using energy saving technologies such as multi-effect evaporators and mechanical vapor recompression, the system is vastly simplified: Firstly, the need for evaporation/condensation is eliminated, which in turn removes the need for large heat exchangers and the energy losses associated with the temperature gradient required to drive the system. Secondly, this invention requires a simpler and less energy-intensive blower, due to the required pressure increase being an order of magnitude lower than that provided by a typical MVR compressor. Thirdly, the entire system may be operated very close to atmospheric pressure, which vastly simplifies construction and increases safety.

### BRIEF DESCRIPTION OF DRAWINGS

FIG. 1 shows the three connected cycles of materials in the system: the tobacco sludge circuit, the steam circuit, and the scrubbing liquid circuit. Additionally shown are: steam heating inputs, and the pressure release valve and its connection to the condenser.

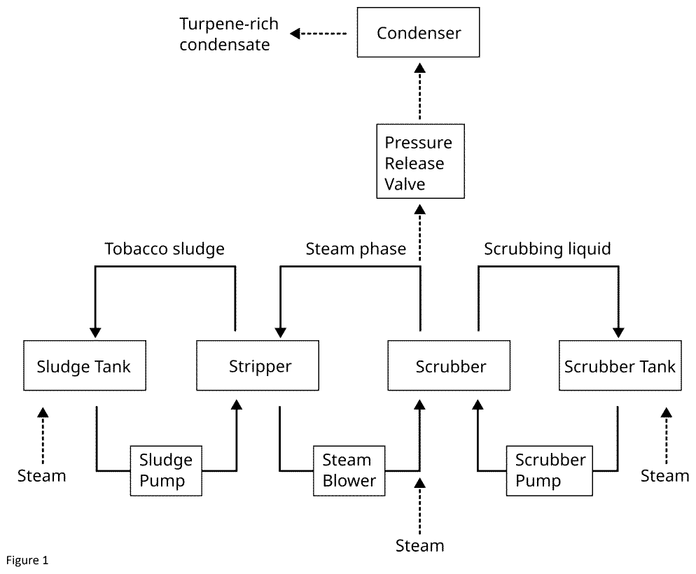

### DETAILED DESCRIPTION OF THE INVENTION

The feedstock, which, when the target alkaloid is nicotine, may be tobacco leaves or any other part of the tobacco plant, or any nicotine-containing product or waste arising from its processing, is first prepared by grinding to a fine powder, most preferably 40 to 80 mesh. The powder is then mixed with enough water to generate a free flowing sludge, most usually 4 to 6 volumes of water. The subsequent sludge is then alkalized through addition of a base such as calcium hydroxide, calcium oxide, sodium hydroxide, potassium carbonate, or any other suitable base, so that the nicotine present is predominantly in the free base form.

The alkalized sludge is then transferred to the sludge tank, and the pump is started to continually transfer the sludge to the stripper, wherein it contacts the steam phase of the system and then returns to the sludge tank.

The scrubber is then prepared by either loading solid or liquid absorbent. In the preferred embodiment, the scrubbing tank is loaded with fresh water, and the pump is started so that the water is continually transported to the scrubber and, after contacting the steam phase, allowed to run back to the tank. A pH regulation system consisting of a pH meter and dosing pump is then started, and automatically adds acid into the scrubbing fluid until the acidity reaches a point where all of the volatile free base nicotine present in the liquid phase has been converted to the non-volatile monoprotonated or di-protonated forms, most usually between pH 3 and 4. Although any water-soluble acid may be used, sulfuric acid is preferred for its low cost, low volatility, and ability to yield nicotine sulfate as a product.

The steam blower is then started, to create a pressure differential and drive the steam phase around the system.

In the simplest embodiment, heating of the system is effected by feeding steam directly from the boiler into the steam circuit until the temperatures of the sludge tank and scrubber liquid tank reach to around 100 C and the pressure release valve is releasing a regular stream of steam. While any method of heating could be used, the direct injection of steam into the vapor circuit is advantageous for its simplicity, while the use of steam heating coils or other heat exchangers in the sludge or scrubber liquid circuits is advantageous as the resultant condensate does not increase the liquid volume of the system.

Additionally, it was found that by connecting the output of the pressure release valve to a water-cooled condenser, it was possible to condense the escaping steam phase to yield a liquid biphase. The organic layer had a strong but pleasant tobacco smell and appeared to be comprised of turpenes, indicating that it may have commercial value for the manufacture of e-liquids or other products requiring a tobacco flavor.

When the process is deemed to be complete (eg. by elapsing of a timer, or by laboratory assay of the sludge, or by in-line UV-VIS analysis of the scrubbing fluid), the pumps and steam blower are turned off. The nicotine-containing scrubber liquid may be removed for further processing, while the substantially nicotine-free sludge may be neutralized by the addition of acid, and used as a suitable feedstock for biogas production. Alternatively, it may be dewatered and used for soil fertilizing, composting, or other agricultural activity.

Although the invention is not restricted to a particular pressure, it is found that limiting operating pressure to slightly above atmospheric pressure decreases equipment cost and increases safety, while keeping the lowest pressure part of the system slightly above atmospheric prevents the ingress of air into the system should there be any leaks. Accordingly, it can be said that, in rough terms, the whole system operates at atmospheric pressure.

Although the invention is not restricted to a particular temperature, operating at the boiling point of water allows for maximum extraction speed. Under these conditions, it can be said that the steam phase approximates pure saturated steam and that the water contents of all three phases (sludge, steam phase, scrubber liquid) are in equilibrium. Accordingly, there is no overall evaporation or condensation of water, hence no associated energy requirements. Instead of the large energy requirements of classic steam distillation or MVR-assisted steam distillation, a vastly reduced amount of power is required to run the steam blower, sludge pump, scrubber pump, and miscellaneous smaller motors/pumps/electronics.

During startup, when the system is cool, the ‘steam phase’ will approximate humid air. As the temperature increases, the gas composition will change from being predominantly air to being predominantly and then almost exclusively steam. As the extraction efficiency appears to be dependent solely on the temperature and not the gas phase composition, and as the gas phase approximates pure saturated steam at target operating point, it is acceptably referred to as the ‘steam phase’ even though it is understood that the invention also encompasses operation at lower temperature regimes where it may be more accurate to refer to the gas phase as being predominantly air.

While the invention is not restricted to a particular method of stripper or scrubber construction, it was found that the key to energy efficiency was to use gas-liquid contactor designs with minimal pressure drops. Additionally, for the stripper, non-fouling construction was found to be essential to avoid material buildup. As gas-liquid contactors usually generate a mist of fine droplets, an integrated gas-liquid separator was found necessary to stop liquid carryover from the scrubber into the stripper, or from the stripper into the scrubber. Preferred contactor types are: venturi contactors, structured packing, random packing, sieve plates and wetted wall contactors. Preferred separator types are: cyclones, knock-out drums, vane packs, and demister pads.

It may be observed that, in the preferred embodiment using dilute sulfuric acid as a scrubber liquid, this method of nicotine recovery yields the nicotine as a nicotine sulfate solution. This is advantageous as it may be further processed toward a commercial 40% solution by simple evaporation, in a completely solvent-free manner.

Although the invention was developed to extract nicotine from tobacco, the principles involved are sufficiently general that many different substances could be extracted from many different plant feedstocks. For example, when using the above-described scrubber, the acidic liquid phase would have a strong affinity for alkaloids such as ephedrine, which may be obtained from plants of the genus Ephedra by steam distillation or solvent extraction.

The invention is also not restricted to plant-based feedstocks and may be useful for the extraction of volatile alkaloids from chemical or biological mixtures during a production process, or for the concentration of dilute alkaloid solutions, or for the purification of impure alkaloids. Examples may include extraction of pseudoephedrine from the L-PAC process, and purification of active pharmaceutical ingredients with volatile free bases, such as salbutamol, epinephrine, and terbutaline.

1. A method of extracting nicotine or other alkaloids from an alkaloid-containing liquid feedstock by continuously passing a steam phase around a closed loop wherein it is alternately contacted with the feedstock, in a gas-liquid contactor known as a stripper, and a material capable of recapturing the target alkaloid from the vapor phase, in a gas-liquid contactor known as a scrubber.

2. The method of claim 1, where the alkaloid is nicotine and/or minor tobacco alkaloids such as nornicotine, anatabine, anabasine, and myosmine, while the feedstock is an alkalized sludge made by mixing water and a base such as calcium hydroxide with nicotine-containing materials such as finely ground tobacco leaf, stems, waste tobacco dust, or other nicotine-containing raw materials, products or waste materials.

3. The method of claim 1, where the material capable of capturing the target alkaloid is activated carbon, an acidic polymer, or, more preferentially, diluted mineral acids such as sulfuric and phosphoric acid.

4. The method of claim 1, where the vapor phase approximates saturated steam at maximum operating temperature, but may consist of various mixtures of air and water vapor at the lower temperatures experienced during startup and shutdown.

5. The method of claim 1, where the stripper and scrubber may be constructed from gas-liquid contactors of the types: venturi contactors, structured packing, random packing, sieve plates and wetted wall contactors, and which may incorporate gas-liquid separators of the types: cyclones, knock-out drums, vane packs, and demister pads.

6. The method of claim 1, where excess vapor pressure may be vented into a condenser to obtain the non-alkaloid portion of the volatile feedstock constituents, for example, turpenes, in the case of tobacco.

7. The method of claim 1, where direct steam injection into the vapor circuit, or heat exchangers in the liquid circuits may be used to raise the temperature of the feedstock and the liquid absorbent to boiling point.

8. The method of claim 1, where the system is operated at around or just over atmospheric pressure, and preferably the minimum increase above atmospheric pressure required to keep the whole system under positive pressure. As the total steam circuit pressure drop is usually less than 3 kPa, setting the pressure release valve to 2˜3 kPa is usually sufficient.

9. The method of claim 1, where the nicotine-depleted feedstock may be used as a feedstock for biogas production.

10. The method of claim 1, where the nicotine-depleted feedstock may be dewatered by using coagulants such as PAC, ferric chloride, ferrous sulfate, and/or flocculants such as PAM, CPAM, APAM, and other neutral/cationic/anionic polymers, in conjunction with dewatering machinery such as filter presses, belt presses, screw presses, and centrifuges, and may then subsequently be used for fertilizing, composting, the growing of mushrooms, or other agricultural activity.
